(19) United States
(12) Patent Application Publication
(10) Pub. No.: US 2021/0186852 A1
(43) Pub. Date: Jun. 24, 2021

(54) Sunscreen and Bug Repellant Formula

(71) Applicant: Louise Dutil, Nipigon (CA)

(72) Inventor: Louise Dutil, Nipigon (CA)

(21) Appl. No.: 17/127,365

(22) Filed: Dec. 18, 2020

Related U.S. Application Data
(60) Provisional application No. 62/951,542, filed on Dec. 20, 2019.

(51) Int. Cl. A61K 8/92; A61Q 17/04; A61Q 17/02; A61Q 17/00; A61K 8/36; A61K 8/9789; A61K 8/9794; A61K 8/9767; A61K 8/9761

(52) U.S. Cl. CPC A61K 8/922; A61Q 17/04; A61Q 17/02; A61Q 17/005; A61K 8/9761; A61K 8/9789; A61K 8/9794; A61K 8/9767; A61K 8/36

(57) ABSTRACT

This present invention relates to a unique topical application that uses natural ingredients to treat or reduce the effects of sunburn and the nuisance from bugs and other biting insects. The formulation includes at least an oil, a vinegar and an essential oil and may be mixed prior to application, or may be pre-mixed and stored for prolonged periods of time. In further embodiments, the formulation may further comprise an anti-microbial/anti-bacterial agent and/or a fragrance. By providing an all-natural product that can address both functions, an individual is not required to purchases multiple products to treat sunburn and insect bites.

### CROSS-REFERENCE TO RELATED APPLICATION

The present application claims priority to, and the benefit of, U.S. Provisional Application No. 62/951,542, which was filed on Dec. 20, 2019 and is incorporated herein by reference in its entirety.

### FIELD OF THE INVENTION

The present invention relates to the field of topical applications for use in preventing sunburn and repelling against insects. More specifically, the formulation of the present invention is made from all-natural ingredients and is an effective sunscreen and insect repellent. Accordingly, the present specification makes specific reference thereto. However, it is to be appreciated that certain aspects of the present invention are also useable with other like applications, devices, and methods of manufacture.

### BACKGROUND OF THE INVENTION

There are currently a large variety of compositions, lotions, sprays, and other products available today for preventing or treating overexposure to the sun's ultraviolet rays, and still other compositions, lotions, sprays, and products for repelling insects, such as mosquitos and the like. Most of these types of products contain various chemicals that create a barrier or other layer on the user's skin that is capable of, for example, shielding the user's skin from over-exposure to the sun, or from insect bites or stings. Unfortunately, such products tend to wear off fairly quickly over time or after being exposed to water, such as in a swimming pool. Once such products wear off or are washed off, they must be reapplied to protect the user, which can be both time consuming, expensive and a general inconvenience.

Additionally, there is a general reluctance on the part of users to repeatedly apply such chemicals onto their skin, which can be harmful over time. Moreover, there is also a general reluctance on the part of users to have such chemicals wash off of the skin and into a natural body of water, such as a river, stream, lake, etc. during swimming, which could negatively impact the environment. In addition, the use of the aerosols and/or propellants needed to force certain chemicals from the canisters in which they are stored is also undesirable as it can put additional chemicals into the atmosphere, as well as be inhaled by the user or those standing near the user during application.

In addition, one normally must purchase multiple products in order to get the benefit of both sunscreen protection and bug repellent. This requires the user having to always stock multiple products and to remember to take the products with them when traveling or going outdoors. Moreover, commercially available products may smell unpleasant, or be unpleasing to the touch (e.g., sticky, oily, etc.) after application to the skin, which is particularly true when an individual has to reapply the composition multiple times during the day to achieve the desired level or protection.

Therefore, there exists a long felt need in the art for a combination product formulation that provides both protection from the sun's harmful rays, and also acts as an insect repellant. There is also a long felt need in the art for a combination product formulation that is all natural, and does not include many of the harmful chemicals currently used in existing sunscreens and insect repellants. Furthermore, there is a long felt need in the art for a combination product formulation that does not require the use of aerosols or propellants during application, which could be harmful to the environment. Moreover, there is a long felt need in the art for a combination product formulation that comprises readily available natural ingredients which may be mixed just prior to application, or which may be pre-mixed and held on the shelf for more than a year before being ready for use. Additionally, there is a long felt need in the art for a combination product formulation that is neither oily or sticky to the touch after application and that emits a neutral or pleasant fragrance. Finally, there is a long felt need in the art for a combination product formulation that is relatively inexpensive to manufacture, and both safe and easy to use.

The combination product formulation of the present invention accomplishes all of the forgoing objectives, and substantially departs from the concepts and designs of the conventional products. The combination product formulation of the present invention may be easily applied by the user, without causing harm to the environment.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The present invention is an all-natural consumer product which provides the user with the dual benefits of sunscreen protection and a bug/insect repellant. The product solution may be distributed in a metered spray amount without the use of aerosols or propellants that may otherwise harm the environment, and the combination solution may be mixed just prior to application or pre-mixed in advance and stored for a period of time depending on the specific shelf life of the particular ingredients used.

In one exemplary embodiment of the presently described invention, a combination topical application is disclosed for providing both sunscreen protection to an individual, wherein the combination topical application also acts as an insect repellant. The combination topical application is preferably comprised of an oil, a vinegar and one or more essential oils that differ from the oil.

In a further exemplary embodiment of the presently described invention, a sunscreen and bug repellant formulation is disclosed. The sunscreen and bug repellant formulation is preferably comprised of a vinegar selected from a group including, but not limited to, white vinegar, distilled vinegar, wine vinegar, cider vinegar and balsamic vinegar, wherein the vinegar component comprises at least 20% of the entire formulation by weight. The sunscreen and bug repellant formulation further comprises an oil that comprises at least 50% of the entire formulation by weight, and an essential oil for providing at least a bug repellent property and selected from a group including a cedarwood, cinnamon, peppermint, citronella, lavender, orange or sweet orange, eucalyptus, spearmint, tea tree, and combinations thereof.

In a yet still further exemplary embodiment of the presently described invention, a sprayable sunscreen and bug repellant composition is disclosed. The sprayable sunscreen and bug repellant composition is capable of being dispersed in metered amounts, and is preferably comprised of an oil, a vinegar, and an essential oil. The oil is provided in an amount ranging from about 20 to 30% by weight of the solution, and the vinegar is provided in an amount ranging from about 60 to 75% by weight of the solution. The essential oil provides sunscreen and bug repellant properties, and is provided in a range of about 1 to 10% of the overall solution by weight.

In a further preferred embodiment, a solution for providing a consumer product with sunscreen and bug/insect repellant characteristics is disclosed. The solution preferably comprises an olive oil in a range from between 20 to 25% by weight of the solution, a white vinegar in a range from between 60 to 75% by weight of the solution, and an essential oil in a range of about 1 to 10% of the solution by weight. It is also contemplated that the solution may further comprise a pleasant or relatively neutral fragrance, other than that which may be supplied by the essential oil and/or an anti-microbial agent, such as an alcohol, or a naturally occurring anti-microbial/anti-bacterial agent like oregano oil.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a spray device that can be used to dispense the combination product formulation of the present invention in accordance with the disclosed architecture; and

FIG. 2 illustrates a perspective view of one potential embodiment of a spray device that can be used to dispense the combination product formulation of the present invention in accordance with the disclosed architecture, wherein the spray device is being used by an individual to apply the combination product formulation.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention or do not limit the scope of the invention. Additionally, an illustrated embodiment need not have all the aspects or advantages shown. Thus, in other embodiments, any of the features described herein from different embodiments may be combined.

As stated above, the combination sunscreen and insect repellant formulation of the present invention is preferably comprised of three main components, namely an oil, a vinegar and one or more essential oils that differ from the oil. Nonetheless, it is also contemplated that the combination sunscreen and insect repellant formulation could also comprise one or more of an anti-microbial/anti-bacterial agent, a fragrance to add a neutral or pleasant overall fragrance to the formulation, or a naturally occurring skin soothing agent, such as aloe, etc. For example, the anti-microbial agent may be used to kill off or prevent harmful germs, bacterial, pathogens and the like from manifesting on the user's skin. In fact, oregano oil is one of the most powerful antibacterial essential oils because it contains carvacrol and thymol, two antibacterial and antifungal compounds which are effective against many clinical strains of bacteria, such as Escherichia coli.

With respect to the oil component, many different types of natural oils may be used as part of the combination sunscreen and insect repellant formulation including, without limitation, vegetable oil, olive oil, canola oil, peanut oil, coconut oil, or combinations thereof. In an exemplary embodiment, olive oil is the preferred oil and may have a grade selected from virgin, extra virgin, or light. Nonetheless, regardless of what type of oil is selected, experimental studies have proven that an oil in the range of about 20 to 30% by weight of the formulation is most effective. The average shelf life of these types of oil is two to four years at room temperature.

The vinegar used in the combination sunscreen and insect repellant formulation of the present invention is preferably one of a white vinegar, distilled vinegar, wine vinegar, cider vinegar or balsamic vinegar. In a preferred embodiment, white vinegar is used. Regardless of which type of vinegar is used, experimental studies have proven that a vinegar in the range of about 60 to 70% by weight of the formulation is most effective. Certain types of vinegar may have an indefinite shelf life, while other vinegars may have a shelf life ranging from two to five years at room temperature.

Various essential oils maybe used in connection with the combination sunscreen and insect repellant formulation of the present invention depending on the specific properties sought from the formulation. For example, some of the essential oils that may be used include, without limitation, lavender, peppermint, lemon, frankincense, lemongrass, clove, cinnamon, cedarwood, sandalwood, vetiver, oregano, grapefruit, eucalyptus, citronella, tea tree, chamomile, geranium, balsam, helichrysum, patchouli, petitgrain, rose, hyssop, rosemary, sage, spearmint, basil, arborvitae, orange, cassia, or combinations thereof.

Essential oils such as cedarwood, cinnamon, peppermint, citronella, lavender, orange or sweet orange, eucalyptus, spearmint, tea tree, and combinations thereof may be selected for use with repelling insects and other bugs. Essential oils such as tea tree, lavender, chamomile, vetiver, geranium, balsam, helichrysum, patchouli, and petitgrain and combinations thereof may be used to sooth the skin and provide a sun screen function in order to protect the skin from sun burn.

The essential oils used in the combination sunscreen and insect repellant formulation of the present invention are provided generally in a range of 1 to 10% of the formulation by weight, and more preferably in the range of 2 to 5% by weight of the formulation. In a further preferred embodiment of the present invention, the essential oil of the combination sunscreen and insect repellant formulation is selected from a tea tree, lavender, or combinations thereof. The average shelf life of essential oils ranges from about 1 to 2 years.

As previously stated, the combination sunscreen and insect repellant formulation of the present invention may be mixed just prior to application, or may be pre-mixed in advance and stored for a period of time depending on the specific shelf life of the particular ingredients used. If the combination sunscreen and insect repellant formulation of the present invention is premixed, the shelf life of the formulation will be largely dependent on the type of essential oil solution that is used, which would provide a minimum shelf life of the solution of about 12 months.

Additionally, the combination sunscreen and insect repellant formulation of the present invention is typically dispensed in the form of a spray, such as through a spray bottle, and without the use of aerosols or propellants during application, which could be harmful to the environment. The droplets of the combination sunscreen and insect repellant formulation of the present invention may be formed in a variety of ways, but generally result from forcing the liquid formulation through a specially designed nozzle assembly. One example of a spray dosage form is a metered-dose topical transdermal spray that delivers a precisely controlled quantity of formulation or suspension on each activation. The particle size contained within such a spray typically ranges from about 1 to 50 microns.

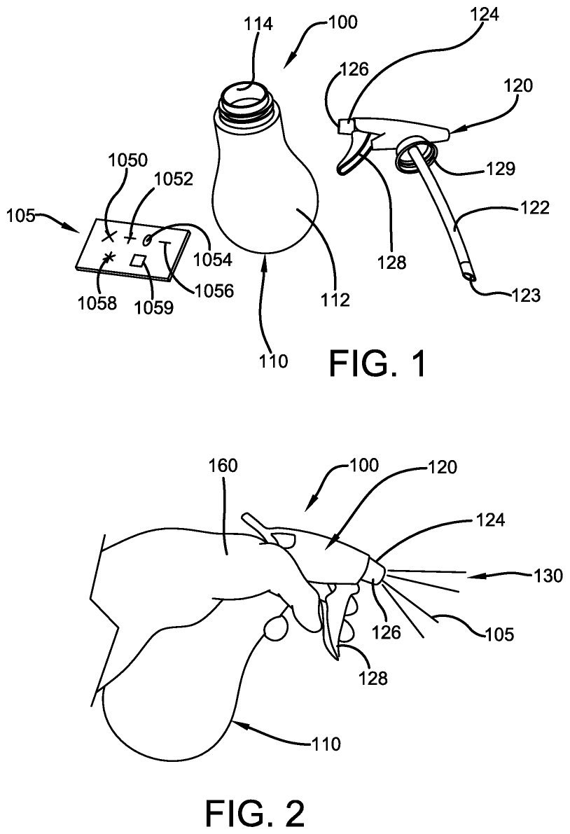

Referring now to the drawings, FIG. 1 illustrates a perspective view of one potential embodiment of a spray device 100 that can be used to dispense the combination sunscreen and insect repellant formulation 105 of the present invention in accordance with the disclosed architecture. The spray device 100 is preferably comprised of a housing 110 and a corresponding trigger assembly 120. The housing 110 defines an interior space 112 for mixing, storing and/or transporting the combination sunscreen and insect repellant formulation 105 of the present invention, and is further comprised of a threaded opening 114 for mating engagement with the trigger assembly. As best shown in FIGS. 1 and 2, the housing 110 is preferably comprised of an easy to handle shape or configuration that is capable of holding between 8 and 16 ounces of the combination sunscreen and insect repellant formulation 105, but the same is not so limited and the housing 110 may be any shape, size or configuration that suits user need and/or preference. Further, the housing 110 is preferably comprised of a durable plastic.

The combination sunscreen and insect repellant formulation 105 may be mixed in the housing 110, or mixed in another container and later poured into the interior space 112 of housing 110. As explained supra, the combination sunscreen and insect repellant formulation 105 may be comprised of any combination of the following: an oil 1050, a vinegar 1052, an essential oil 1054, an anti-bacterial agent 1056, a fragrance 1058 and/or a skin soothing agent 1059. Nonetheless, in a preferred embodiment, the combination sunscreen and insect repellant formulation 105 will comprise at least an oil 1050, a vinegar 1052, and an essential oil 1054 in the percentages by weight referenced above.

The trigger assembly 120 is preferably comprised of a straw or hose 122, a nozzle 124 having an opening 126 therein, a trigger 128 and a connector 129. The straw or hose 122 is in fluid communication with both the formulation 105 stored in the interior space 112 of the housing 110 and the opening 126 in the nozzle 124 when the trigger 128 is activated and, accordingly, extends downwardly from the rest of the trigger assembly 120 into the formulation 105. More specifically, the trigger 128, when activated by a user, acts like a pump and draws the formulation 105 through the straw 122, nozzle 124 and propels the same outwardly through the opening 126 and onto the user's skin in a measured dose. The connector 129 is preferably threaded to correspond to the threaded opening 114 of the housing to removably secure the trigger assembly 120 to the housing in a watertight fashion.

FIG. 2 illustrates a perspective view of one potential embodiment of a spray device 100 that can be used to dispense the combination product formulation 105 of the present invention in accordance with the disclosed architecture, wherein the spray device 105 is being used by an individual 160 to spray 130 the combination product formulation 105.

Certain terms are used throughout the following description and claims to refer to features or components. As one skilled in the art will appreciate, different persons may refer to the same feature or component by different names. This document does not intend to distinguish between components or features that differ in name but not structure or function.

Notwithstanding the forgoing, the components of the spray device 100 can be of any suitable size and configuration as is known in the art without affecting the overall concept of the invention, provided that it accomplishes the above stated objectives. Furthermore, one of ordinary skill in the art will appreciate that the formulation and composition are for illustrative purposes only, and that many other components and ingredients are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A topical application comprising;
an oil;
a vinegar; and
an essential oil, wherein the topical application is both a sunscreen and an insect repellant.

2. The topical application as recited in claim 1, wherein the oil is one of a vegetable oil, an olive oil, a canola oil, a peanut oil, a coconut oil or a combination thereof.

3. The topical application as recited in claim 1, wherein the oil is an olive oil and has a grade selected from one of a virgin grade, an extra virgin grade or a light grade.

4. The topical application as recited in claim 1, wherein the vinegar is selected from one of a white vinegar, a distilled vinegar, a wine vinegar, a cider vinegar, a balsamic vinegar or a combination thereof.

5. The topical application as recited in claim 1, wherein the vinegar is a white vinegar.

6. The topical application as recited in claim 1, wherein the essential oil is selected from a group consisting or a lavender, a peppermint, a lemon, a frankincense, a lemongrass, a clove, a cinnamon, a cedarwood, a sandalwood, a vetiver, an oregano, a grapefruit, an eucalyptus, a tea tree, a chamomile, a geranium, a balsam, a helichrysum, a patchouli, a petitgrain, a rose, a hyssop, a rosemary, a sage, a spearmint, a basil, a citronella, an arborvitae, an orange, a cassia or a combination thereof.

7. The topical application as recited in claim 1, wherein the topical application is comprised of between 20 and 25 percent of the oil by weight, 60 and 75 percent of the vinegar by weight, between 2 and 3% of the essential oil by weight.

8. The topical application as recited in claim 1, wherein the topical application is comprised of an olive oil that comprises 25 percent by weight of the topical application, between 70 and 75 percent of a white vinegar by weight, and between 2 and 3 percent by weight of the essential oil.

9. The topical application as recited in claim 1 further comprising an anti-bacterial agent.

10. The topical application as recited in claim 1 further comprising a fragrance.

11. The topical application as recited in claim 1, wherein the topical application is applied to a user's skin in a spray that has a particle size ranging from about 1 to 50 microns.

12. The topical application as recited in claim 11, wherein the spray does not include an aerosol.

13. A sunscreen and bug repellant formulation comprising;
at least 20% by weight of a vinegar selected from a group including a white vinegar, a distilled vinegar, a wine vinegar, a cider vinegar and a balsamic vinegar;
at least 50% by weight of an oil; and
an essential oil for providing at least a bug repellent property selected from a group including a cedarwood, a cinnamon, a peppermint, a citronella, a lavender, an orange or a sweet orange, an eucalyptus, a spearmint, a tea tree, and a combination thereof.

14. The sunscreen and bug repellant formulation as recited in claim 13, wherein the essential oil further includes a sunscreen property and is selected from a group including a tea tree, a lavender, a chamomile, a vetiver, a geranium, a balsam, a helichrysum, a patchouli, a petitgrain and a combination thereof.

15. The sunscreen and bug repellant formulation as recited in claim 13, wherein the sunscreen and bug repellant formulation is distributed in a metered amount in a spray that has a particle size ranging from about 1 to 50 microns and further wherein the spray does not include an aerosol.

16. A sprayable sunscreen and bug repellant composition comprising:
an oil provided in an amount ranging from about 20 to 30 percent by weight of the sprayable sunscreen and bug repellant composition;
a vinegar provided in an amount ranging from about 60 to 75 percent by weight of the sprayable sunscreen and bug repellant composition;
an anti-microbial; and
an essential oil provided in an amount ranging from about 1 to 10 percent by weight of the sprayable sunscreen and bug repellant composition, wherein the essential oil provides sunscreen and bug repellant properties, and further wherein the sprayable sunscreen and bug repellant composition is capable of being distributed in a metered amount.

17. The sprayable sunscreen and bug repellant composition as recited in claim 16, wherein the essential oil is selected from a tea tree or a lavender.

18. The sprayable sunscreen and bug repellant composition as recited in claim 16, wherein the essential oil is selected from a group consisting of a lavender, a peppermint, a lemon, a frankincense, a lemongrass, a clove, a cinnamon, a cedarwood, a sandalwood, a vetiver, an oregano, a grapefruit, an eucalyptus, a tea tree, a chamomile, a geranium, a balsam, a helichrysum, a patchouli, a petitgrain, a rose, a hyssop, a rosemary, a sage, a spearmint, a basil, a citronella, an arborvitae, an orange, a cassia, or a combination thereof.

19. The sprayable sunscreen and bug repellant composition as recited in claim 16, wherein the vinegar is selected from one of a white vinegar, a distilled vinegar, a wine vinegar, a cider vinegar and a balsamic vinegar.

20. The sprayable sunscreen and bug repellant composition as recited in claim 16, wherein the oil is one of a vegetable oil, an olive oil, a canola oil, a peanut oil, a coconut oil or a combination thereof.
